(19) United States
(12) Patent Application Publication
(10) Pub. No.: US 2008/0112967 A1
(43) Pub. Date: May 15, 2008

(54) Process for Refining Ganoderma Spore Polysacchoride

(75) Inventors: Peng Feng, Nanjing (CN); Min Feng, Nanjing (CN); Jing Su Huang, Nanjing (CN); Yi Fan Qian, Nanjing (CN); Jian Shen, Nanjing (CN); Liang Chen, Nanjing (CN); Lian An Wang, Nanjing (CN)

Correspondence Address: GLOBAL IP SERVICES, 7285 W. Eagle Court, Winton, CA, 95388, US

(73) Assignee: NANJING ZHONGKE GROUP CORP., LTD., Nanjing (CN)

(21) Appl. No.: 11/563,769

(22) Filed: Nov. 28, 2006

(30) Foreign Application Priority Data
Oct. 25, 2006 (CN) 200610097229.7

(51) Int. Cl. A61K 36/06; C12P 19/04

(52) U.S. Cl. 424/195.16; 536/123

(57) ABSTRACT

The present invention discloses a process for refining Ganoderma spore polysacchoride. The process comprise the step of: Remove the impurity in the Ganoderma spores, warm water extraction, alcohol precipitation, separate precipitate, obtain the impure polysacchoride, adjust pH to 7.5-8.5, centrifuge to removal deposit, take out supernatent, add H₂O₂ to decolor, deproteined by sevag method, dialyses, alcohol precipitation, wash the precipitate, dry in the vacuum to get the pure Ganoderma spore polysacchoride.

### CROSS REFERENCE TO RELATED PATENT APPLICATION

This patent application claims the priority benefit of a Chinese patent application No. 200610097229.7 filed on Oct. 25, 2006.

### FIELD OF THE INVENTION

The present invention belongs to the field of Chinese traditional medicine, and it relates to the process for refining Ganoderma spore polysacchoride.

### BACKGROUND OF THE INVENTION

Ganoderma is the encarpium of Ganoderma, Fomes japonica, etc. They are all eumycetes which belong to Polyporaceae. Ganoderma's flavour is sweet and its nature is plain. The function is supplementing qi and blood, calming the mind of heart, strengthening the spleen and stomach. It begins to be recorded as a high grade from the Shennong's Classic of Materia Medica. It is praised that has several kinds of functions, such as benefiting qi of the heart, supplementing the stomach, increasing the intelligence, protecting the spirit, profiting the essence, smoothing the joints, firming the bones and muscles and the person would felt light of body and be macrobiosis after eating it. Generally speaking, even multiple diseases recorded by ancient books of the traditional Chinese medicine in the past dynasties, they all belong to the diseases due to the deficient and strain body. It indicates that reinforcing the deficiency and making the body strong is Ganoderma's speciality. This is exactly same as the theory of the modern pharmacology which realizing Ganoderma can improve the nonspecific immunity capability and reinforce disease resistance of the body.

Spore powder of Ganoderma is the spore punched out from the mature Ganoderma. Along with the improving of modern technology methods, it has a new recognition to the spore of Ganoderma. After being destroyed the paries, it was developed into a new health-protective remedy. It condenses the essence of Ganoderma and receives the right qi of the sky and earth. So it is pharmacodynamic action and clinical application effects have more obvious enhancement.

Ganoderma Spore contains chemical substances, e.g. proteins, amino acids, polyose glycopeptides, adenosines, sterols, triterpenes, alkaloids, fatty acids and the like. Modern pharmacology and clinical trial confirm, comparing with the Ganoderma Lucidum, Ganoderma Spore powder have more strong and comprehensive effect, Ganoderma Spore powder can anti-chromosomal mutate, induce tumor cell death, decrease tumor cell proliferation, kill cancer cell, prevent the side effect caused by antagonism endoxan, e.g. the decrease in the stamina, chromosome aberration the decrease in the hemoglobin and leucocyte, the decrease in the macrophage phagocytic power and SGPT increasing and the like, prevent the side effect of leukopenia decreasing caused by ⁶⁰Co irradiation, active and increase the antineoplastic action of the specificity killer cell (CTL) and non-specificity killer cell (NK, LAK, increase monocyte macrophage phagocytic power, prevent lipotropism peroxidation and the like, it shows the fineness potential of the Ganoderma spore in regulating immunologic system, resisting tumour.

Ganoderma spore is manufactured by refining Ganoderma Lucidum, broking seedcase, shattering, then pelletizing and fat removing. Then polysacchoride is obtained by extracting what is got in the above-mentioned process by water, depositing by alcohol and drying. Polysacchoride is one of the effective constituent of the Ganoderma spore. It is appeared in the experiment that Ganoderma spore polysacchoride can active mouse macrophage, Ganoderma spore polysacchoride has obvious antineoplastic curative effect, it can increase Lewis lung cancer mouse NK activity.

A process for abstracting Ganoderma spore polysacchoride is disclosed in the CN1483743. In the process, firstly, it uses CO₂ supercritical extraction method to extract Ganoderma spore oil for another application, and makes the residue obtained after the ganoderma spore oil is extracted undergo the processes of removing fat, water extraction, alcohol precipitation, filtration and dryness so as to obtain the Ganoderma spore polysacchoride. The process utilizes Ganoderma spore powder what is removed fat, it makes polysacchoride extraction easy, and polysacchoride content increased. However, the polysacchoride obtained in this process is still not pure, in the medicine research, it only can be used in the manufacture of oral preparation, and it can't produce injection preparation, and in this process, it need to boil and reflux for abstracting for 1-10 h, however, the temperature is high in the polysacchoride extraction(>90° C.), the extraction time is long (>1 h), it may destroy the structure of polysacchoride, it may even crack the 5C-ring and 6C-ring, and it change the molecular configuration, and the structure is not exact.

### SUMMARY OF THE INVENTION

The object of the invention is to provide a process for refining Ganoderma spore polysacchoride.

The object of the invention is solved by:

a) Remove the impurity in the Ganoderma spores, break seedcase, removed fat;

b) warm water extraction: put the 1 g residue obtained after the ganoderma spore oil is extracted into 10-20 g water, extract 0.5-2.5 h at the temperature of 70-90° C., repeat 1-3 times, mix the extracting solution, leave stand, filter and condense the filtrate;

c) Alcohol precipitation: precipitated by 85-95% ethanol until the ethanol content is 75-85%, leave stand, separate precipitate, washed by 85-100% ethanol, dried, then the impure polysacchoride is gained;

d) refine: resolve the impure polysacchoride in the suitable water, adjust pH to 7.5-8.5, centrifuge to removal deposit, take out supernatent, add H₂O₂ to decolor, deproteined by sevag, dialyses, add 95% ethanol until the ethanol content is 70-90%, alcohol precipitation, wash the precipitate by absolute ethanol and acetone 2-3 times in turn, dry in the vacuum, then, the pure Ganoderma spore polysacchoride is gained.

The above-mentioned process for refining Ganoderma spore polysacchoride, preferably comprises the step of:

a) Remove the impurity in the Ganoderma spores, break seedcase, shattered, then pelletized and use CO₂ supercritical extraction method to extract Ganoderma spore oil to remove fat;

b) warm water extraction: put the 1 g residue obtained after the ganoderma spore oil is extracted into 10-20 g water, extract 0.5-2.5 h at the temperature of 70-90° C., repeat 1-3 times, mix the extracting solution, leave stand, filter and condense the filtrate;

c) Alcohol precipitation: precipitated by 85-95% ethanol until the ethanol content is 75-85%, leave stand, separate precipitate, washed by 85-100% ethanol, dried, then the impure polysacchoride is gained;

d) refine: resolve the impure polysacchoride in the suitable water, adjust pH to 7.5-8.5, centrifuge to removal deposit, take out supernatent, add H₂O₂ to decolor, deproteined by sevag method, dialyses, add 95% ethanol until the ethanol content is 70-90%, alcohol precipitation, wash the precipitate by absolute ethanol and acetone 2-3 times in turn, dry in the vacuum, then, the pure Ganoderma spore polysacchoride is gained.

In the above-mentioned process for refining Ganoderma spore polysacchoride, it utilizes CO₂ supercritical extraction method to extract Ganoderma spore oil, its process parameters: extract temperature: 35˜65° C., extract pressure: 20˜35 Mpa, extract time: 2˜7 h, CO₂ flow rate: 0.5-1 m³/h.

Advantageous Effects of the Invention:

In the above-mentioned process, it use warm water extraction method to extract polysacchoride, the destruction to the polysacchoride is decreased, and it use H₂O₂ to decolor, use sevag method to deprotein, use alcohol precipitation to produce pure Ganoderma spore polysacchoride, it has the advantage of low cost and good effect.

Sample: the sample of No.20040101, 20040102, 20040103 is impure polysacchoride produced by the method of example 1 (procedure a-c); the sample of no.20040101p, 20040102p, 20040103p is pure polysacchoride produced by the method of example 1 (procedure a-d).

1) The research of process for refining Ganoderma spore polysacchoride

The common process for refining polysacchoride comprise the step of: depigmentation, deproteinization, alcohol precipitation, dehydrate to dry and the like, it is complex. It is reported in the literature that the precipitation method of metal complex method, quaternary ammonium salt precipitation method and the like to precipite polysacchoride, the pigment and protein is still in the solvent, it can omit the steps of depigmentation and deproteinization. However, in the refining process, both of methods can't precipite Ganoderma spore polysacchoride. Thus, polysacchoride is puried by common methods.

1 Depigmentation

1.1 Active Carbon Decolor Method

Take the three samples (No. 20040101, 20040102, 20040103), each samples is 10 g, add 400 ml water into the samples, heat the samples to it solve in the water, add ammonia to adjust pH to 7.8, centrifuge at 4000 rpm. Put the precipitation into 0.2 mol/l HCl and solve the precipitation (the precipitation can be solved in the water, it especially can be solved in the acid easily), add the Coomassie brilliant blue reagent into it, the solution appears blue, it proved that it is protein; take out the supernatant, add 60 ml 30% H₂O₂ into it (15 vol % of polysaccharide solution), keep the temperature at 40° C., when the solution is turned from brown to straw yellow, cooled to room temperature.

2 Deproteinization

In the deproteining method, we compare the Sevag method and TCA (trichloroacetic acid) method. In the TCA method, no protein precipitated. Before the solution of Ganoderma spore polysaccharide is decolored by with H₂O₂, most of the protein can be removed by add ammonia to adjust pH to 7.8, the remained protein is removed by Seveg method.

Seveg method: take the polysaccharide solution decolored, add the chloroform-butanol (4:1) solution which has the same volume, then vibrate it, lay it overnight, remove the underlayer gel substance, take the superstrata solution and deal it with the same method until underlayer solution do not appearance gel substance no longer. Put the polysaccharide solution which the protein is removed completed into bag filter and flow the water to dialyse for 24 h. The remained solution (dialyse solution) is condensed to ⅓ volume of the original solution.

3 Alcohol Precipitation, Dryness

Put the condensed polysaccharide solution into absolute ethanol to precipitate polysaccharide, lay in the refrigerator overnight, filter it by No. 4 sand core funnel, wash the precipitation with absolute ethanol and acetone for three times, dry in the vacuum, the white pure Ganoderma spore polysaccharide is obtained, weigh the product and calculate the yield.

The three Ganoderma spore polysaccharide raw material (No. 20040101, 20040102, 20040103) is decolored by active carbon, deproteined by Sevag method, alcohol precipitate and dry to get straw yellow pure Ganoderma spore polysaccharide the yield is 39.67%, 43.09%, 46.10%. And the white pure Ganoderma spore polysaccharide (No. 20040101p, 20040102p, 20040103p) produced by decoloring with H₂O₂, deproteining with Sevag method, alcohol precipitating and drying, their yield are 63.67%, 59.09%, 67.70%.

Take the pure polysaccharide which is produced by making the same Ganoderma spore polysaccharide (No. 20040101) as raw material, using active carbon and H₂O₂ to decolor, use the HPLC (high performance liquid chromatograph: HP1050, America: chromatographic column: Shodex Ohpak SB-805 HQ, 300 mm×8 mm, Japan Showa denko kk; mobile phase: double distraction water; velocity of flow: 0.6 ml/min; evaporation light scattering detector (ELSD) SEDEX 75, France; ELSD detector drift tube temperature: 50° C.; ELSD detector pressure: 3.5 bar; ELSD detector gain value: 7; chromatogram workstation: jiangshen JS-3050 type chromatogram workstation (bring GPC software); Dextran standard molecular weight polysaccharide: Fluka corporation. Add the obtained pure polysaccharide into water to solve the polysaccharide, and produce 5 mg/ml solution, after high speed centrifugation, use the 0.45 μm millipore filter to filtrate the supernatant and take 20μ 1 to inject chromatographic column, record chromatogram) to compare the chromatogram of two pure polysaccharide, it is obvious that, the two components is nearly same, it means that decoloration with H₂O₂ would not destroy the structure of Ganoderma spore polysaccharide. The decoloration is very difficult in filtrating, the decoloration is not enough, and the loss of polysaccharide is more. So in the present invention, the decoloration method use H₂O₂ to decolor.

4 The Protein Examination

4.1 Coomassie Brilliant Blue Method

Add 0.5 g pure Ganoderma spore polysaccharide in the 10 ml measure bottle, add water to solve the polysaccharide and add water to dilute to the solstice scale, shake it up. Filtrate the solution by paper filter, take 60 ml filtrate; take 30μ 1 mg/ml BSA, and add 30 μl water to dilute it, take the diluent as reference solution, add 3 ml Coomassie brilliant blue test solution to each solution, shake the solution up and lay at the room temperature for 15 min, begin the colorimetric estimation at 595 nm. Compare the sample solution and the reference solution. In the three pure product (No. 20040101p, 20040102p, 20040103p), the content of protein is no more than 1%. The result is shown in table 1.

TABLE 1
the result of protein examination

| Measuration | | | | |
|---|---|---|---|---|
| solution | BAS | 20040101p | 20040102p | 20040103p |
| concentration(μg/μl) | 1.02 | 49.92 | 50.01 | 50.23 |
| sampling | 30 | 60 | 60 | 60 |
| quantity(μl) | | | | |
| absorbance | 0.445 | 0.358 | 0.370 | 0.372 |
| protein(%) | | 0.82 | 0.85 | 0.85 |

4.2 UV Method

Take some pure Ganoderma spore polysaccharide, add water to produce 1 mg/ml solution, begin ultraviolet scan at 200˜400 nm, the three pure product (No.20040101p, 20040102p, 20040103p) is not detected the characteristic absorption peak of nucleic acid (260 nm) and protein (280 nm).

2) The Pharmacodynamics Test

(1) The inhibitory action of reference Ganoderma spore polysaccharide to animal transplantation tumor

1 The Test Material

1.1 The test medicine: the reference Ganoderma spore polysaccharide (it is named No. 1), it is produced by the method disclosed in the CN1483743, the specific steps is: add Ganoderma spore powder into distilled water, the added amount can shape the Ganoderma spore, shake it up, pelletize, dry in the air, use CO₂ supercritical extraction method to remove fat, weigh 10 kg Ganoderma spore which is removed fat, add it into the abstraction pot, add 150 Kg water, soak it for 2 h, heat to boil, keep reflux for 4 h, centrifuge the extracting solution, condense the filtrate to 5 Kg, add three times 95% ethanol, lay it 24 h, filter the polysaccharide precipitation, dry the precipitation so as to obtain the Ganoderma spore polysaccharide.

1.2 Animal:ICR mouse, 18-22 g, half male and half female, the mouses is provided by China drug section university animal laboratory. The number of certificate of conformity is: SCXK (SU)2002-0011. The feedstuff is diet pellet, it is provided by China drug section university animal laboratory. Breeding condition: air conditioning room, Temperature 18-24° C., Relative humidity: 70%.

1.3 Masculine Drug: endoxan (CTX), HENGRUI MEDICINE CO LTD JIANG. Specification: 200 mg/bottle, Batch Number: 06060121.

2 The Primary Coverage of the Experimental Study

2.1 the inhibitory action of the No. 1 oral pour stomach administration to the mouse transplantation tumor Heps.

2.1 the inhibitory action of the No. 1 oral pour stomach administration to the mouse transplantation tumor S₁₈₀.

3 Experimental Method and Step

3.1 the inhibitory action of the No. 1 oral pour stomach (ip) administration to the mouse transplantation tumor Heps.

3.1.1 administration route: oral pour stomach (ip)

3.1.2 administration period: Inoculate Heps entity type by transplantation tumor methodology, after inoculating 24 h, administrate to the mouse and ip administrate, once a day, administrate for 7 times, at the next day when the administration is stopped, kill the mouse.

3.1.3 dosage, there are four groups:

blank reference group(normal saline)

No. 1: 160 mg/kg

No. 1: 40 mg/kg

CTX: 20 mg/kg

3.1.4 experimental method: Take 40 above-mentioned mouses (Heps entity type inoculated by transplantation tumor methodology), after the mouse is inoculated, weigh the mouse, and divide them into 4 groups, there is 10 mouses in every groups, half male and half female, blank reference group and CTX group are negative and masculine reference group. After inoculating 24 h, administrate to the mouse and ip administrate, once a day, administrate for 7 times, at the next day when the administration is stopped, weigh the mouses and kill them, weigh the weight of tumor, statisticaly treat the obtained data (t verify).

3.1.5 Result

The result is shown in table 2, it is revealed that, No. 1 (160 mg/kg, 40 mg/kg) can inhibit the growth of Heps tumor in the mouse (P<0.01,P<0.05) compared with the blank reference group.

TABLE 2
the inhibition action of No. 1 to the transplation
tumor of Heps mouse (X ± SD)(n = 10)

| | | | | | The rate of |
|---|---|---|---|---|---|
| | | Weight(g) | | the weight of | inhibiting |
| | dosage | Before | After | tumor | tumor |
| group | (mg/kg) | anministration | anministration | (g) | (%) |
| reference group | | 20.2 ± 1.40 | 27.8 ± 2.44 | 1.61 ± 0.51 | |
| No. 1 | 160 | 20.5 ± 1.27 | 23.0 ± 2.24** | 1.02 ± 0.30** | 36.58 |
| | 40 | 20.6 ± 1.43 | 23.89 ± 1.27** | 1.28 ± 0.35 | 20.77 |
| CTX | 30 | 20.1 ± 1.30 | 24.3 ± 2.41** | 0.43 ± 0.15** | 64.60 |

*P < 0.05
**P < 0.01 compared with blank reference group

3.2 the inhibitory action of the No. 1 oral pour stomach (ip) administration to the mouse transplantation tumor S₁₈₀.

3.2.1 administration route: oral pour stomach (ip)

3.2.2 administration period: Inoculate S₁₈₀ entity type by transplantation tumor methodology, after inoculating 24 h, administrate to the mouse and ip administrate, once a day, administrate for 7 times, at the next day when the administration is stopped, kill the mouse.

3.2.3 dosage, there are four groups:

blank reference group(normal saline)

No. 1: 160 mg/kg

No. 1: 40 mg/kg

CTX: 20 mg/kg

3.2.4 experimental method: Take 40 above-mentioned mouses (S₁₈₀ entity type inoculated by transplantation tumor methodology), after the mouse is inoculated, weigh the mouse, and divide them into 4 groups, there is 10 mouses in every groups, half male and half female, blank reference group and CTX group are negative and masculine reference group. After inoculating 24 h, administrate to the mouse and ip administrate, once a day, administrate for 7 times, at the next day when the administration is stopped, weigh the mouses and kill them, weigh the weight of tumor, statisticaly treat the obtained data t verify.

3.2.5 Result

The result is shown in table 3, it is revealed that, No. 1 (160 mg/kg, 40 mg/kg) can inhibit the growth of S₁₈₀ tumor in the mouse (P<0.01,P<0.05) compared with the blank reference group.

TABLE 3
the inhibition action of No. 1 to the transplation
tumor of S180 mouse (X ± SD)(n = 10)

| | | | | | The rate of |
|---|---|---|---|---|---|
| | | Weight(g) | | the weight of | inhibiting |
| | dosage | Before | After | tumor | tumor |
| group | (mg/kg) | anministration | anministration | (g) | (%) |
| reference group | | 20.1 ± 1.37 | 27.1 ± 2.13 | 1.43 ± 0.23 | |
| No. 1 | 160 | 20.5 ± 1.35 | 24.1 ± 1.90** | 0.95 ± 0.37** | 33.55 |
| | 40 | 20.6 ± 0.97 | 26.0 ± 1.76 | 0.84 ± 0.21* | 21.49 |
| CTX | 30 | 20.1 ± 0.94 | 24.3 ± 1.68** | 0.51 ± 0.18** | 65.81 |

P < 0.05
**P < 0.01 compared with blank reference group

(2) The Inhibitory Action of Ganoderma Spore Polysaccharide of the Present Invention to Animal Transplantation Tumor

1 The Test Material

1.1 The test medicine: the Ganoderma spore polysaccharide of the present invention (produced by the method of the present invention, it is named No.2.).

1.2 Animal: ICR mouse, 18-22 g, half male and half female, the mouses is provided by China drug section university animal laboratory. The number of ertificate of conformity is: SCXK(SU)2002-0011. The feedstuff is diet pellet, it is provided by China drug section university animal laboratory. Breeding condition: air conditioning room, Temperature 18-24° C., Relative humidity: 70%.

1.3 Masculine drug: endoxan (CTX), HENGRUI MEDICINE CO LTD JIANG.

Specification: 200 mg/bottle, Batch Number: 06060121.

2 The Primary Coverage of the Experimental Study

2.1 the inhibitory action of the No. 2 intravenous injection administration to the mouse transplantation tumor Heps.

2.1 the inhibitory action of the No. 2 intravenous injection administration to the mouse transplantation tumor S₁₈₀.

3 Experimental Method and Step

3.1 the inhibitory action of the No. 2 intravenous injection (iv) administration to the mouse transplantation tumor Heps.

3.1.1 administration route: intravenous injection (iv)

3.1.2 administration period: Inoculate Heps entity type by transplantation tumor methodology, after inoculating 24 h, administrate to the mouse and ip administrate, once on alternate days, administrate for 4 times, at the next day when the administration is stopped, kill the mouse.

3.1.3 dosage, there are four groups:

blank reference group (normal saline)

No. 2: 3 mg/kg

No. 2: 1 mg/kg

CTX: 20 mg/kg 3.1.4 experimental method: Take 40 above-mentioned mouses (Heps entity type inoculated by transplantation tumor methodology), after the mouse is inoculated, weigh the mouse, and divide them into 4 groups, there is 10 mouses in every groups, half male and half female, blank reference group and CTX group are negative and masculine reference group. After inoculating 24 h, administrate to the mouse and ip administrate, once on alternate days, administrate for 4 times, at the next day when the administration is stopped, weigh the mouses and kill them, weigh the weight of tumor, statistically treat the obtained data (t verify).

3.1.5 Result

The result is shown in table 4, it is revealed that, No. 2 (3 mg/kg, 1 mg/kg) can inhibit the growth of Heps tumor in the mouse (P<0.01,P<0.05) compared with the blank reference group, it may decreas the weight of the mouse, but it has little influence compared with CTX, and the effort of No. 2 is advantageous compared with No. 1.

TABLE 4
the inhibition action of No. 2 to the transplation
tumor of Heps mouse (X ± SD)(n = 10)

| | | | | | The rate of |
|---|---|---|---|---|---|
| | | Weight(g) | | the weight of | inhibiting |
| | dosage | Before | After | tumor | tumor |
| group | (mg/kg) | anministration | anministration | (g) | (%) |
| blank reference | | 20.2 ± 1.40 | 27.8 ± 2.44 | 1.61 ± 0.51 | |
| group | | | | | |
| No. 2 | 3 | 20.1 ± 1.29 | 23.6 ± 1.90** | 0.80 ± 0.22** | 50.50 |
| | 1 | 20.0 ± 1.76 | 24.8 ± 2.62* | 1.12 ± 0.37* | 30.44 |
| CTX | 30 | 20.1 ± 1.30 | 24.3 ± 2.41** | 0.43 ± 0.15** | 64.60 |

*P < 0.05
**P < 0.01 compared with blank reference group

3.2 the inhibitory action of the No. 2 intravenous injection (iv) administration to the mouse transplantation tumor S₁₈₀.

3.2.1 administration route: intravenous injection (iv)

3.2.2 administration period: Inoculate S₁₈₀ entity type by transplantation tumor methodology, after inoculating 24 h, administrate to the mouse and ip administrate, once on alternate days, administrate for 4 times, at the next day when the administration is stopped, kill the mouse.

3.2.3 dosage, there are four groups:

blank reference group (normal saline)

No. 1: 3 mg/kg

No. 1: 1 mg/kg

CTX: 20 mg/kg

3.2.4 experimental method: Take 40 above-mentioned mouses (S₁₈₀ entity type inoculated by transplantation tumor methodology), after the mouse is inoculated, weigh the mouse, and divide them into 4 groups, there is 10 mouses in every groups, half male and half female, blank reference group and CTX group are negative and masculine reference group. After inoculating 24 h, administrate to the mouse and ip administrate, once on alternate days, administrate for 4 times, at the next day when the administration is stopped, weigh the mouses and kill them, weigh the weight of tumor, statisticaly treat the obtained data t verify.

3.2.5 Result

The result is shown in table 4, it is revealed that, No.2 (3 mg/kg, 1 mg/kg) can inhibit the growth of Heps tumor in the mouse (P<0.01,P<0.05) compared with the blank reference group, it may decreas the weight of the mouse, but it has little influence compared with CTX, and the effort of No.2 is advantageous compared with No. 1.

TABLE 5
the inhibition action of No. 2 to the transplation
tumor of S₁₈₀ mouse (X ± SD)(n = 10)

| | | | | | The rate of |
|---|---|---|---|---|---|
| | | Weight(g) | | the weight of | inhibiting |
| | dosage | Before | After | tumor | tumor |
| group | (mg/kg) | anministration | anministration | (g) | (%) |
| reference group | | 20.1 ± 1.37 | 27.1 ± 2.13 | 1.43 ± 0.23 | |
| No. 1 | 3 | 20.9 ± 1.60 | 24.9 ± 2.81 | 0.85 ± 0.36** | 40.48 |
| | 1 | 20.6 ± 1.58 | 25.4 ± 1.71 | 1.09 ± 0.36* | 23.80 |
| CTX | 30 | 20.1 ± 0.94 | 24.3 ± 1.68** | 0.51 ± 0.18** | 65.81 |

P < 0.05
**P < 0.01 compared with blank reference group

### BRIEF DESCRIPTION OF THE DRAWING

FIG. 1 is the chromatogram comparison diagram of the Ganoderma spore polysaccharide decolored by active carbon and H₂O₂. In the Figure, ts.c.sla represents the pure Ganoderma spore polysaccharide decolored by active carbon, ts.sys.sla represents the pure Ganoderma spore polysaccharide decolored by H₂O₂.

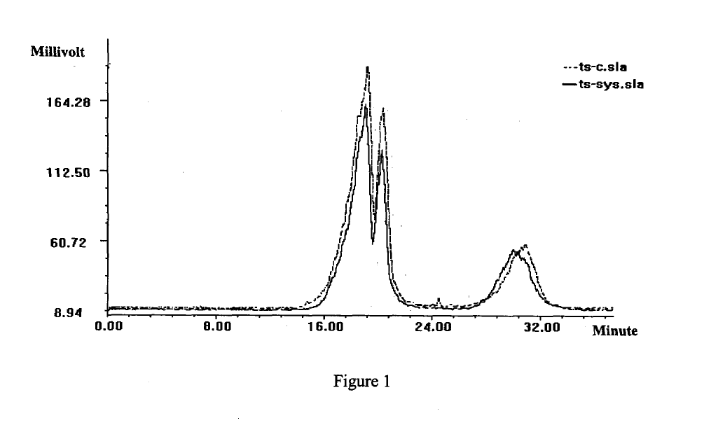

### DETAILED DESCRIPTION OF THE INVENTION

Hereinafter, it will further detail the present invention through the example.

### EXAMPLE 1

Remove the impurity in the Ganoderma spores, break seedcase and add distilled water, after the spores are broken, the spore powder and the distilled water are 1:0.25 in weight, pelletize, dry at the temperature below 60° C. for 4 h, until the content of water is below 5%, put the dried Ganoderma spore powder into extractor, inlet CO₂ from the bottom of the extractor, the extracting temperature is 60° C., the pressure is 26 MPa, the extracting time is 5 h, the flow of CO₂ is 0.6 m³/h; take the Ganoderma spore oil out and put them into decompression segregator, Ganoderma spore oil are flowed out from the bottom of decompression segregator, CO₂ are flowed out from the top of decompression segregator; separate and remove the water in the Ganoderma spore oil to get the pure Ganoderma spore oil for another application, take out the Ganoderma spore powder from extractor; add water to the Ganoderma spore powder removed fat, extract the powder at 90° C. for 3 times, each time is 1.5 h, mix the extraction, lay the solution 24 h, filtrate and decompression condense to the ⅙ volume of the original product; precipitate with 95% ethanol until the content of ethanol is 80%, lay at 4° C. for 16 h; take out the supernatant, recover ethanol, wash the precipitation with 95% ethanol for 3 times, evaporate the ethanol in the air, dry at the temperature below 60° C. to obtain the impure polysacchoride; put the polysacchoride into water to obtain 10% polysacchoride solution, add ammonia to adjust pH to 7.8, centrifuge to remove deposit, decolor the supernatant with H₂O₂, deprotein with seveg method, dialyses, precipitate with 85% alcohol, wash the precipitation with absolute alcohol and acetone 2-3 times in turn, dry in the vacuum at the low temperature, then, the pure Ganoderma spore polysacchoride is gained. The polysacchoride content is 93.2% by anhydrous glucose (C₆H₁₂O₆).

### EXAMPLE 2

Remove the impurity in the Ganoderma spores, break seedcase and pelletize, dry at the temperature below 60° C. for 4 h, put the dried Ganoderma spore powder into extractor, inlet CO₂ from the bottom of the extractor, the extracting temperature is 35° C., the pressure is 35 MPa, the extracting time is 4 h, the flow of CO₂ is 0.5 m³/h; take the Ganoderma spore oil out and put them into decompression segregator, Ganoderma spore oil are flowed out from the bottom of decompression segregator, CO₂ are flowed out from the top of decompression segregator; separate and remove the water in the Ganoderma spore oil to get the pure Ganoderma spore oil for another application, take out the Ganoderma spore powder from extractor; add water to the Ganoderma spore powder removed fat, extract the powder at 70° C. for 3 times, each time is 1.5 h, mix the extraction, lay the solution 24 h, filtrate and decompression condense to the ⅙ volume of the original product; precipitate with 95% ethanol until the content of ethanol is 80%, lay at 0° C. for 16 h; take out the supernatant, recover ethanol, wash the precipitation with 95% ethanol for 3 times, evaporate the ethanol in the air, dry at the temperature below 60° C. to obtain the impure polysacchoride; put the polysacchoride into water to obtain 12% polysacchoride solution, add ammonia to adjust pH to 7.8, centrifuge to remove deposit, decolor the supernatant with H₂O₂, deprotein with seveg method, dialyses, precipitate with 85% alcohol, wash the precipitation with absolute alcohol and acetone 2-3 times in turn, dry in the vacuum at the low temperature, then, the pure Ganoderma spore polysacchoride is gained. The polysacchoride content is 87.1% by anhydrous glucose (C₆H₁₂O₆).

### EXAMPLE 3

Remove the impurity in the Ganoderma spores, break seedcase and pelletize, dry at the temperature below 60° C. for 4 h, put the dried Ganoderma spore powder into extractor, inlet CO₂ from the bottom of the extractor, the extracting temperature is 65° C., the pressure is 20 MPa, the extracting time is 3 h, the flow of CO₂ is 1 m³/h; take the Ganoderma spore oil out and put them into decompression segregator, Ganoderma spore oil are flowed out from the bottom of decompression segregator, CO₂ are flowed out from the top of decompression segregator; separate and remove the water in the Ganoderma spore oil to get the pure Ganoderma spore oil for another application, take out the Ganoderma spore powder from extractor; add water to the Ganoderma spore powder removed fat, extract the powder at 80° C. for 2 times, each time is 1.5 h, mix the extraction, lay the solution 24 h, filtrate and decompression condense to the ⅙ volume of the original product; precipitate with 95% ethanol until the content of ethanol is 80%, lay at 0° C. for 15 h; take out the supernatant, recover ethanol, wash the precipitation with 95% ethanol for 3 times, evaporate the ethanol in the air, dry at the temperature below 60° C. to obtain the impure polysacchoride; put the polysacchoride into water to obtain 15% polysacchoride solution, add ammonia to adjust pH to 7.8, centrifuge to remove deposit, decolor the supernatant with H2O2, deprotein with seveg method, dialyses, precipitate with 85% alcohol, wash the precipitation with absolute alcohol and acetone 2-3 times in turn, dry in the vacuum at the low temperature, then, the pure Ganoderma spore polysacchoride is gained. The polysacchoride content is 73.6% by anhydrous glucose (C₆H₁₂O₆).

### EXAMPLE 4

Remove the impurity in the Ganoderma spores, break seedcase and pelletize, dry at the temperature below 60° C. for 4 h, put the dried Ganoderma spore powder into extractor, inlet CO₂ from the bottom of the extractor, the extracting temperature is 60° C., the pressure is 28 MPa, the extracting time is 4 h, the flow of CO₂ is 0.8 m³/h; take the Ganoderma spore oil out and put them into decompression segregator, Ganoderma spore oil are flowed out from the bottom of decompression segregator, CO₂ are flowed out from the top of decompression segregator; separate and remove the water in the Ganoderma spore oil to get the pure Ganoderma spore oil for another application, take out the Ganoderma spore powder from extractor; add water to the Ganoderma spore powder removed fat, extract the powder at 90° C. for 2 times, each time is 0.5 h, mix the extraction, lay the solution 24 h, filtrate and decompression condense to the ⅙ volume of the original product; precipitate with 95% ethanol until the content of ethanol is 80%, lay at 4° C. for 18 h; take out the supernatant, recover ethanol, wash the precipitation with 95% ethanol for 3 times, evaporate the ethanol in the air, dry at the temperature below 60° C. to obtain the impure polysacchoride; put the polysacchoride into water to obtain 10% polysacchoride solution, add ammonia to adjust pH to 7.8, centrifuge to remove deposit, decolor the supernatant with H2O2, deprotein with seveg method, dialyses, precipitate with 85% alcohol, wash the precipitation with absolute alcohol and acetone 2-3 times in turn, dry in the vacuum at the low temperature, then, the pure Ganoderma spore polysacchoride is gained. The polysacchoride content is 69% by anhydrous glucose (C₆H₁₂O₆).

1. A process for refining Ganoderma spore polysaccharide comprising steps of:
a) removing mixed impurity in Ganoderma spores, breaking and shattering seedcase of the Ganoderma spores to get spore powder;
b) removing fat, using CO₂ supercritical extraction method to extract Ganoderma spore oil from the spore powder to remove fat;
c)warming water extraction: put 1 g residue of the spore powder obtained after Ganoderma spore oil is extracted into 10-20 g water, extract 0.5-2.5 h at temperature of 70-90° C., repeat 1-3 times, mix the extractive solution, keep the mixed extractive solution at rest, filter the extractive solution and condense the filtrate;
d) alcohol precipitation: the condensed extracting solution got from step c) is precipitated by 85-95% ethanol until the ethanol content is 75-85%, keep the precipitated solution at rest, separate precipitate, wash the precipitate by 85-100% ethanol, dry the washed precipitate, then impure polysaccharide is gained;
e) refining: resolve the impure polysaccharide in water, adjust the solution pH to 7.5-8.5, centrifuge to remove deposit, take out supernatant, decolor the supernatant by adding H₂O₂ , deproteinate the decolored supernatant by sevag method, dialyses, add 95% ethanol until the ethanol content is 70-90%, get alcohol precipitate, wash the precipitate by absolute ethanol and acetone 2-3 times in turn, dry the washed precipitate in the vacuum, then, pure Ganoderma spore polysaccharide is gained.

2. The process for refining Ganoderma spore polysaccharide according claim 1, wherein:
a) Removing the mixed impurity in the Ganoderma spores, breaking and shattering the seedcase of the Ganoderma spores, then palletizing;
b) removing fat: using CO₂ supercritical extraction method to extract Ganoderma spore oil from the spore power to remove fat;
c) warming water extraction: put the 1 g residue of the spore powder obtained after the ganoderma spore oil is extracted into 10 water, extract 1.5 h at the temperature of 90° C., repeat 1-3 times, mix the extractive solution, keep the mixed extractive solution at rest, filter the extractive solution and condense the filtrate;
d) alcohol precipitation: the condensed extracting solution got from step c is precipitated by 95% ethanol until the ethanol content is 80%, keep the precipitated solution at rest, separate precipitate, wash the precipitate by 95% ethanol, dry the washed precipitate, then the impure polysaccharide is gained;
e) refining: resolve the impure polysaccharide in the suitable water, adjust pH to 7.8, centrifuge to removal deposit, take out supernatant, add H₂O₂ to decolor, deproteined by sevag method, dialyses, add 95% ethanol until the ethanol content is 85%, alcohol precipitation, wash the precipitate by absolute ethanol and acetone 3 times in turn, dry in the vacuum, then, the pure Ganoderma spore polysaccharide is gained.

3. The process for refining Ganoderma spore polysaccharide according claim 1, wherein said CO₂ supercritical extraction method to extract Ganoderma spore oil is under process parameters: extract temperature: 35˜65° C., extract pressure: 20˜35 Mpa, extract time: 2˜7 h, CO₂ flow rate:0.5-1m³/h.

4. The process for refining Ganoderma spore polysaccharide according claim 2, wherein the CO₂ supercritical extraction method to extract Ganoderma spore oil is under process parameters: extract temperature: 35˜65° C., extract pressure: 20˜35 Mpa, extract time: 2˜7 h, CO₂ flow rate:0.5-1m³/h.

5. The process for refining Ganoderma spore polysaccharide according claim 2, wherein the process parameters of the CO₂ supercritical extraction method to extract Ganoderma spore oil are as follows: extract temperature: 35˜65° C., extract pressure: 20˜35 Mpa, extract time: 2˜7 h, CO₂ flow.
